(19) United States
(12) Patent Application Publication
(10) Pub. No.: US 2022/0025341 A1
(43) Pub. Date: Jan. 27, 2022

(54) MINIMAL PEPTIDE FUSIONS FOR TARGETED INTRACELLULAR PROTEIN DEGRADATION

(71) Applicant: Massachusetts Institute of Technology, Cambridge, MA (US)

(72) Inventors: Pranam CHATTERJEE, Cambridge, MA (US); Joseph M. JACOBSON, Newton, MA (US)

(21) Appl. No.: 17/334,055

(22) Filed: May 28, 2021

Related U.S. Application Data
(60) Provisional application No. 63/032,513, filed on May 29, 2020.

(51) Int. Cl. C12N 9/10; A61P 31/14; C12N 9/48

(52) U.S. Cl. CPC C12N 9/104; A61P 31/14; A61K 38/00; C12N 9/48; C12Y 304/17023; C12Y 203/02

(57) ABSTRACT

Methods and compositions relating to an engineered peptide capable of binding to an infectious biological molecule for inhibition by mediated degradation using the ubiquitin proteasome pathway. The engineered peptide includes a targeting domain and an ubiquitin ligase recruiting domain. The engineered peptide includes a targeting domain and an ubiquitin ligase. The targeting domain is computationally-derived from a known receptor for the infectious biological molecule. The engineered peptide is optimized for minimal size and minimum off-target effects.

### CROSS-REFERENCE TO RELATED APPLICATION

The present application claims the benefit of U.S. Provisional Application No. 63/032,513, filed May 29, 2020, which is hereby incorporated by reference in its entirety.

### FIELD OF TECHNOLOGY

The present disclosure relates to competitive peptides, particularly fusion peptides for targeting viral and other infectious microbes for intracellular degradation, more particularly minimal fusion peptides with an ubiquitin ligase and a targeting domain binding to a SARS-CoV-2 spike protein receptor.

### BACKGROUND

SARS-CoV-2 has emerged as a highly pathogenic coronavirus and has now spread to over 200 countries, infecting over 50 million people worldwide and killing over 1 million people as of October 2020. Economies have crashed, travel restrictions have been imposed, and public gatherings have been canceled, all while a sizeable portion of the human population remains quarantined. Rapid transmission dynamics as well as a wide range of symptoms, from a simple dry cough to pneumonia and death, are common characteristics of coronavirus disease 2019 (COVID-19) [Wu, J. T. et al. Estimating clinical severity of COVID-19 from the transmission dynamics in Wuhan, China. Nat. Med. 26, 506-510 (2020).]. With no cures readily available [Lurie, N., Saville, M., Hatchett, R. & Halton, J. Developing covid-19 vaccines at pandemic speed. N. Engl. J. Med. 382, 1969-1973 (2020)], and only limited vaccine availability, there is a pressing need for robust and effective therapeutics targeting the virus.

Numerous antiviral strategies have been proposed to limit SARS-CoV-2 replication by preventing viral infection and synthesis [Senanayake, S. L. Drug repurposing strategies for COVID-19. Future Drug Discov. 2 (2020).]. As SARS-CoV-2 is a positive-sense RNA virus, Abbott, et al. recently devised a CRISPR-Cas13d based strategy, termed PAC-MAN, to simultaneously degrade the positive-sense genome and viral mRNAs [Abbott, T. R. et al. Development of CRISPR as an antiviral strategy to combat SARS-CoV-2 and influenza. Cell 181, 865-876.e12 (2020)]. While this method may serve as a potential prophylactic treatment, introducing foreign and relatively large components such as Cas13 enzymes into human cells in vivo presents various delivery and safety challenges [Lino, C. A., Harper, J. C., Carney, J. P. & Timlin, J. A. Delivering CRISPR: a review of the challenges and approaches. Drug Deliv. 25, 1234-1257 (2018).].

Targeted protein depletion is a key method of disrupting protein-protein interactions and protein function in vivo. Protein synthesis can be blocked at various levels. At the DNA level, protein coding genes can be disrupted using genome editing tools, such as zinc-finger nucleases, TALENs, and CRISPR-Cas9 [Gaj, et al., “ZFN, TALEN, and CRISPR/Cas-based methods for genome engineering”, Trends Biotechnol. (2013)]. At the post-transcriptional level, methods such as RNAi or CRISPR-Cas13 can be used for degrading targeted messenger RNAs (mRNAs) [Boettcher, et al., “Choosing the right tool for the job: RNAi, TALEN or CRISPR”, Mol. Cell (2015)]. Finally, at the translational level, antisense oligonucleotides can be utilized to hybridize to the mRNA and block the progression of the translation initiation complex from the 5′ cap to the start codon [Eisen, J. S. et al., “Controlling morpholino experiments: don't stop making antisense”, Development (2008)].

The most rapid and acute method of protein degradation intracellularly, however, is at the post-translational level. Specifically, E3 ubiquitin ligases can tag endogenous proteins for subsequent degradation in the proteasome [Ardley, et al., “E3 ubiquitin ligases”, Essays Biochem. (2005)]. Thus, by guiding E3 ubiquitin ligases to a protein of interest, one can mediate its depletion.

Numerous previous works have attempted to redirect E3 ubiquitin ligases by replacing their natural protein binding domains with those targeting specific proteins [Gosink, M. M. et al., “Redirecting the specificity of ubiquitination by modifying ubiquitin-conjugating enzymes”, PNAS (1995); Zhou, et al., “Harnessing the ubiquitination machinery to target the degradation of specific cellular proteins”, Mol. Cell (2000); Su, et al., “Eradication of pathogenic β-catenin by Skp1/Cullin/F box ubiquitination machinery”, PNAS (2003)]. Recently, the TRIM-Away method was devised, wherein antibodies targeting specific proteins would be recognized, via their Fc domain, by the exogenously expressed human TRIM 21 E3 ubiquitin ligase, thus facilitating rapid and acute protein degradation [Clift, et al., “Acute and rapid degradation of endogenous proteins by Trim-Away”, Cell (2018)].

Previous works have attempted to redirect E3 ubiquitin ligases by replacing their natural protein binding domains with those targeting specific proteins. In 2014, Portnoff, et al. reprogrammed the substrate specificity of a modular human E3 ubiquitin ligase called CHIP (carboxyl-terminus of Hsc70-interacting protein) by replacing its natural substrate-binding domain with designer binding proteins to generate optimized “ubiquibodies” or uAbs. [Portnoff, A. D., Stephens, E. A., Varner, J. D. & DeLisa, M. P. Ubiquibodies, synthetic e3 ubiquitin ligases endowed with unnatural substrate specificity for targeted protein silencing. J. Biol. Chem. 289, 7844-7855 (2014).]

The COVID-19 pandemic, caused by the novel coronavirus SARS-CoV-2, has elicited a global health crisis of catastrophic proportions. With only a few vaccines approved for early or limited use, there is a critical need for effective antiviral strategies.

### SUMMARY

The present invention includes peptide-Fc and peptide-E3 ubiquitin ligase fusions that represent a minimal protein to proteasome linkers. These fusions can mediate targeted and robust degradation of expressed intracellular proteins with expression of a cognate E3 ubiquitin ligase.

In some aspects, the engineered peptide for mediated degradation of a target infectious microbe comprises a fusion protein comprising a targeting domain and a ubiquitin ligase recruiting domain, wherein the targeting domain is engineered to bind the target infectious microbe for mediation degradation by the ubiquitin-proteosome pathway.

In some aspects, the target infectious microbe is a virus.

In some aspects, the targeting domain binds to a spike protein receptor. In some aspects, the spike protein receptor is part of a viral envelope. In some aspects, the spike protein receptor is part of a coronavirus. In some aspects, the coronavirus is SARS-CoV-2.

In some aspects, the targeting domain comprises an amino acid sequence selected from the group consisting of SEQ. ID. 1 through SEQ. ID. 3. In some aspects, the targeting domain comprises an amino acid sequence is at least 90% identical to a sequence selected from the group consisting of SEQ. ID. 1 through SEQ. ID. 3. In some aspects, the targeting domain comprises an amino acid sequence is at least 80% identical to a sequence selected from the group consisting of SEQ. ID. 1 through SEQ. ID. 3.

In some aspects, the ubiquitin ligase recruiting domain is an Fc domain. In some aspects, the viral receptor binding domain has a C-terminus, and the Fc domain is fused to the C-terminus. In some aspects, the ubiquitin ligase recruiting domain recruits an E3 ubiquitin ligase. In some aspects, the E3 ubiquitin ligase is TRIM21, corresponding to SEQ. ID. 4.

In some aspects, the ubiquitin ligase recruiting domain is an E3 ubiquitin ligase. In some aspects, the E3 ubiquitin ligase is CHIPΔTPR, corresponding to SEQ. ID. 5.

In some aspects, the method for the treatment or alleviation of an infection by an infectious microbe in a subject comprises administering to the subject an engineered peptide or pharmaceutically acceptable salt thereof, wherein the engineered peptide comprises a fusion of a targeting domain and a ubiquitin ligase recruiting domain and wherein the targeting domain is engineered to bind the target infectious microbe for mediation degradation by the ubiquitin-proteosome pathway.

In some aspects, the targeting domain comprises an engineered sACE2-derived peptide.

In some aspects, the targeting domain comprises an amino acid sequence selected from the group consisting of SEQ. ID. 1 through SEQ. ID. 3. In some aspects, the targeting domain comprises an amino acid sequence is at least 90% identical to a sequence selected from the group consisting of SEQ. ID. 1 through SEQ. ID. 3.

In some aspects, the infection is caused by a coronavirus.

### BRIEF DESCRIPTION OF THE DRAWINGS

Other aspects, advantages and novel features of the invention will become more apparent from the following detailed description of the invention when considered in conjunction with the accompanying drawings.

FIG. 1 is a diagram of mediated degradation of a virus by recruitment of a E3 ubiquitin ligase by a fusion protein, according to certain embodiments of the present disclosure.

FIG. 2 is a graph demonstrating the efficacy of the example mediated degradation of FIG. 1, according to certain embodiments of the present disclosure.

FIG. 3 is a diagram of mediated degradation of a virus by a fusion protein with a E3 ubiquitin ligase, according to embodiments of the present disclosure.

FIG. 4A is a schematic representation of an example experimental outline for testing efficacy of the example mediated degradation of FIG. 1 using the SARS-CoV-2 receptor binding domain (RBD) fused to GFP and tested for GFP degradation, according to certain embodiments of the present disclosure.

FIG. 4B is a schematic representation of an example experimental outline for testing efficacy of the example mediated degradation of FIG. 3 using the SARS-CoV-2 RBD fused to GFP and tested for GFP degradation, according to certain embodiments of the present disclosure.

FIG. 5 is a graph demonstrating the efficacy of the example experimental outline of FIG. 4, according to certain embodiments of the present disclosure.

### DETAILED DESCRIPTION

The object of the present disclosure relates to mediated degradation of an infectious microbes using fusion peptides for targeting the infectious microbes for intracellular degradation.

The object of the present disclosure particularly relates to mediated degradation of antiviral molecules, and more particularly relates to computational design of fusion peptides for the mediation of viral degradation. The present disclosure particularly relates to engineered fusion peptides for targeting SARS-CoV-2 for mediation degradation by the ubiquitin-proteosome pathway.

The present disclosure includes peptide-Fc and peptide-E3 ubiquitin ligase fusions that represent a minimal protein to proteasome linker. These fusion peptides can mediate targeted and robust degradation of expressed intracellular proteins with expression of a cognate E3 ubiquitin ligase.

In some aspects, the present invention includes utilization of a peptide that binds a protein of interest in a human cell. The target-binding peptide is either directly fused to the human Fc domain and co-expressed with an E3 ubiquitin ligase, such as TRIM 21, which will lead to ubiquitination and degradation of the protein of interest. In certain preferred embodiments, the target-binding peptide is fused directly to any E3 Ubiquitin Ligase, such as CHIPΔTPR, TRIM 21, or any E3 ubiquitin ligase.

Further disclosed is an optimized peptide variant that enables robust degradation of RBD complexes in human cells, both in trans and in cis with human E3 ubiquitin ligases. The fusion constructs disclosed herein inhibit the production of infection-competent viruses pseudotyped with the full-length S protein of SARS-CoV-2.

In some circumstances, mediation of degradation by a single construct may be desired. To engineer a single construct that can mediate SARS-CoV-2 degradation without trans expression of an E3 ubiquitin ligase, the RBD-binding proteins can be fused to an E3 ubiquitin ligase, such as the CHIPΔTPR modified E3 ubiquitin ligase domain.

Herein, a targeted intracellular degradation strategy for SARS-CoV-2 is disclosed by computationally designing peptides that bind to its spike (S) protein receptor binding domain (RBD) and recruit an E3 ubiquitin ligase for subsequent proteasomal degradation. The disclosed results of example experiments identify effective peptide variants that mediate robust degradation of the RBD fused to a stable superfolder-green fluorescent protein (sfGFP) in human cells and demonstrates inhibition of infection-competent viral production [Pédelacq, J. D., Cabantous, S., Tran, T., Terwilliger, T. C. & Waldo, G. S. Engineering and characterization of a superfolder green fluorescent protein. Nat. Biotechnol. 24, 79-88 (2005).].

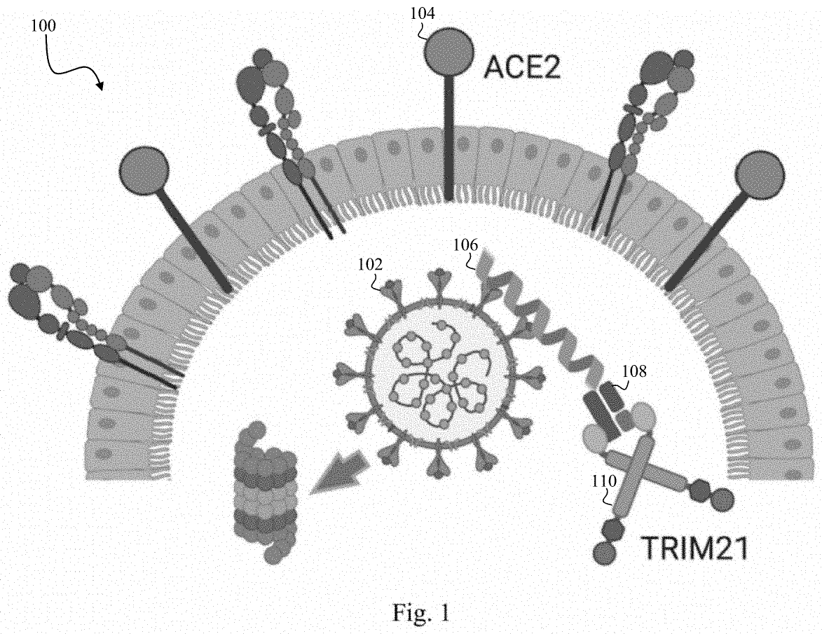

Referring now to FIG. 1, an example mediated degradation 100 of a SARS-CoV-2 virus is shown to demonstrate the function of the fusion peptide in mediating degradation of a target virus. As an example of developing peptide fusions to proteins within human cells, the spike protein receptor binding domain (RBD) 102 of the SARS-CoV-2 virus can be targeted for degradation. The fusion peptide of example 100 comprises a targeting domain 106 fused to a Fc domain 108. The Fc domain 108 recruits an E3 ubiquitin ligase 110, such as TRIM 21 as shown in example 100.

Targeting domain 106 may be computationally designed to target a particular virus, such as SARS-CoV-2, by being configured to bind to a known, conserved binding domain of the target virus, such as the RBD 102 of example 100. In embodiments, targeting domain 106 may be derived from known binding partner of viral RBD. For example, when targeting the spike protein RBD 102 of a corona virus, targeting domain 106 can be derived from human ACE2.

ACE2 is a monocarboxypeptidase, widely known for cleaving various peptides within the renin-angiotensin system [Tipnes, et al., “A human homolog of angiotensin-converting enzyme: cloning and functional expression as a captopril-insensitive carboxypeptidase”, Journal of Biological Chemistry (2000)]. Functionally, there are two forms of ACE2. The full-length ACE2 104 contains a structural transmembrane domain, which anchors its extracellular domain to the plasma membrane [Du, et al., “The spike protein of sars-cov—a target for vaccine and therapeutic development”, Nat. Rev. Microbiol. (2009)]. The extracellular domain has been demonstrated as a receptor for the spike (S) protein of SARS-CoV, and recently, for the SARS-CoV-2.

The soluble form of ACE2 lacks the membrane anchor, thus preserving binding capacity, and circulates in small amounts in the blood [Wysocki, et al. “Targeting the degradation of angiotensin ii with recombinant angiotensin-converting enzyme 2: Prevention of angiotensin ii-dependent hypertension”, Hypertension (2010)]. Overexpression of soluble ACE2 (sACE2) may act as a competitive interceptor of SARS-CoV-2 and other coronaviruses by preventing binding of the viral particle to the endogenous ACE2 transmembrane protein.

sACE2, however, is capable of binding other biological molecules in vivo, such as integrins [Clarke, et al., “Angiotensin Converting Enzyme (ACE) and ACE2 Bind Integrins and ACE2 Regulates Integrin Signaling”, PLOS ONE (2012)]. It is therefore an object of the present disclosure to ensure therapeutics targeting SARS-CoV-2 epitopes withstand the possibility of viral mutation, which may allow the virus to overcome the host adaptive immune response [Andersen, K. G., Rambaut, A., Lipkin, W. I., Holmes, E. C. & Garry, R. F. The proximal origin of SARS-CoV-2. Nat. Med. 26, 450-452 (2020)]. Embodiments of the present disclosure relate to engineering minimal sACE2 peptides, such as by in silico protein modeling, that not only maintain potent RBD binding, but also possess reduced off-target interaction with the integrin α5β1 receptor.

Furthermore, sACE2 preserves the peptidase activity of its transmembrane counterpart, and its overexpression can thus interfere with pathways dependent on this function, such as insulin/Akt signaling [Zhong, et al., “Enhanced angiotensin converting enzyme 2 regulates the insulin/Akt signalling pathway by blockade of macrophage migration inhibitory factor expression”, Br. J. Pharmacol. (2008)]. High-throughput in silico truncations to the N-terminal peptidase domain of ACE2, the human receptor for SARS-CoV-2, can be conducted, such as with the Rosetta protein modeling software [Rohl, et al., “Protein structure prediction using Rosetta”, Methods Enzymol. (2004)]. The minimized ACE2 protein structure 106 comprises sufficient components needed for binding to the RBD.

The Peptidrive algorithm [Sedan, et al., “Peptiderive server: derive peptide inhibitors from protein-protein interactions”, Nucleic Acids Research (2016)] can be applied one or more times for each peptide to find candidates derived from ACE2 which bind to the spike protein with high affinity. In embodiments, a length between 30 and 100 amino acids may be preferable for candidate peptides. Each candidate protein can be computationally relaxed, and those with the lowest total energy score, and thus highest binding affinity, may be selected for experimental analysis.

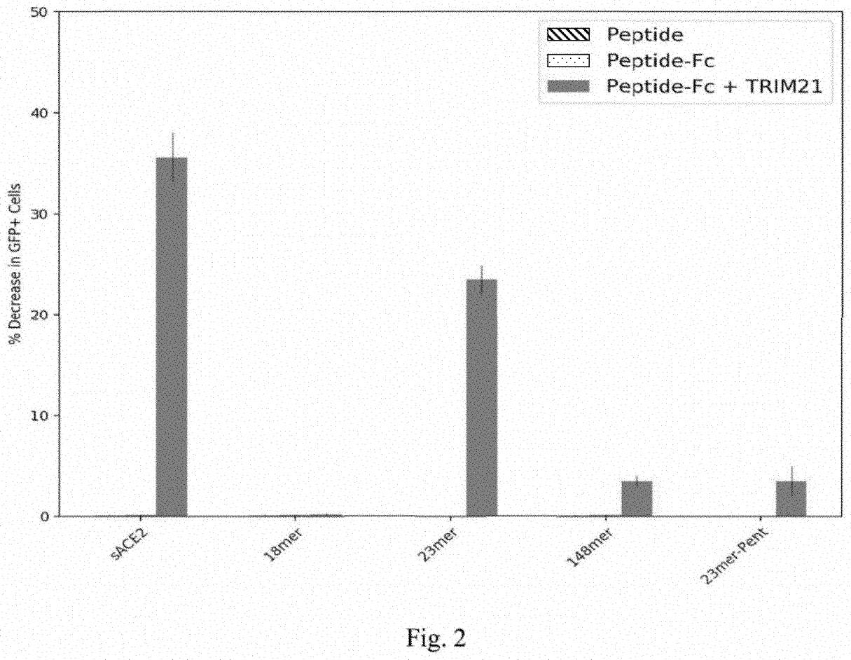

To verify efficacy, sACE2 can be expressed, as well as other candidate sACE2-derived peptides, C-terminally fused to the human Fc receptor 108, as well as the human TRIM 21 protein 110. The results in this example, shown in the graph of FIG. 2, demonstrate that both sACE2 and the candidate 23-mer peptide are capable of mediating degradation of RBD when fused to the Fc domain and expressed alongside TRIM 21.

This disclosure contemplates the usage of any peptide sequence, in conjunction with sequences that are already existent in nature/literature. Defining a peptide as any amino acid sequence shorter than 200 amino acids, example peptide sequences within the scope of the invention include, but are not limited to:

TABLE 1
Example targeting domain peptide sequences

| Seq. ID. | | Derived | |
|---|---|---|---|
| No. | Length | from: | Sequence |
| 01 | 23mer | sACE2 | QAKTFLDKFNHEAEDLFYQSSLA |
| 02 | 23mer | A2N | QNKTFLDKFNHEAEDLFYQSSLA |
| 03 | 23mer | A2N_H11A | QNKTFLDKFNAEAEDLFYQSSLA |

Other targeting domain peptide sequences include, but are not limited to, sequences derived by the method disclosed in U.S. patent application Ser. No. 17/222,676 directed to “Computationally-Optimized ACE2 Peptides for Competitive Interception of SARS-CoV-2,” the entirety of which is incorporated herein by reference.

Methods and products according to the present disclosure have numerous advantages, including but not limited to, both peptide and E3 ubiquitin ligase components can be engineered from endogenous human proteins, which may reduce the risk of immunogenicity.

Also, the peptide fusion platform as a prophylactic provides a viable alternative to current antiviral strategies being explored for COVID-19 and other viruses. Antiretroviral protease inhibitors for HIV, such as lopinavir and ritonavir, have shown minimal efficacy in clinical trials of COVID-19, and generated adverse effects in a subsection of patients [Cao, B. et al. A trial of lopinavir-ritonavir in adults hospitalized with severe covid-19. N. Engl. J. Med. 382, 1787-1799 (2020).]. Similarly, antimalarials, such as hydroxychloroquine and chloroquine, which may glycosylate ACE2, have demonstrated no benefit in patients infected with SARS-CoV-2 in randomized, controlled studies [Boulware, D. R. et al. A randomized trial of hydroxychloroquine as postexposure prophylaxis for covid-19. N. Engl. J. Med. 383, 517-525 (2020).]. A standard timeframe to fully assess safety and efficacy of a vaccine takes well over one year [Callaway, E. The race for coronavirus vaccines: a graphical guide. Nature 580, 576-577 (2020).].

The platform of the present disclosure provides a rapid and direct targeting mechanism, which coupled with its size and human-protein derivation, presents numerous advantages as compared with existing strategies. The strategy of utilizing a computationally designed peptide binder linked to an E3 ubiquitin ligase can be effective not only for SARS-CoV-2, but also for other viruses and drug targets that have known binding partners. With already over 30,000 co-crystal structures currently in the PDB, and structure determination becoming more routine with advances in cryogenic electron microscopy, the computational peptide engineering pipeline presented here provides a versatile new therapeutic platform in the fight against COVID-19, future emergent viral threats, and numerous diseases.

In one aspect, this disclosure includes a method of using any short peptide (such as the examples listed in Table 1), fused to either the Fc domain of antibodies, or any E3 Ubiquitin ligase, to degrade any protein of interest in a human cell. For peptides fused to Fc, an E3 Ubiquitin ligase that can be co-expressed is TRIM 21.

The peptide can be directly fused to the ANY E3 Ubiquitin ligase [Medvar B, Raghuram V, Pisitkun T, Sarkar A, Knepper M A, “Comprehensive database of human E3 ubiquitin ligases: application to aquaporin-2 regulation. Physiol Genomics. 2016;48(7):502□512]. In an example embodiment, an E3 Ubiquitin ligase that can be used is CHIPΔTPR.

TABLE 2
Example E3 Ubiquitin Ligase Sequences

| SEQ. | | |
|---|---|---|
| ID. | | |
| No. | Name | Sequence |
| 04 | TRIM21 | MASAARLTMMWEEVTCPICLDPFVEPVSIECGHSF |
| | | CQECISQVGKGGGSVCPVCRQRFLLKNLRPNRQLA |
| | | NMVNNLKEISQEAREGTQGERCAVHGERLHLFCEK |
| | | DGKALCWVCAQSRKHRDHAMVPLEEAAQEYQEKLQ |
| | | VALGELRRKQELAEKLEVEIAIKRADWKKTVETQK |
| | | SRIHAEFVQQKNFLVEEEQRQLQELEKDEREQLRI |
| | | LGEKEAKLAQQSQALQELISELDRRCHSSALELLQ |
| | | EVIIVLERSESWNLKDLDITSPELRSVCHVPGLKK |
| | | MLRTCAVHITLDPDTANPWLILSEDRRQVRLGDTQ |
| | | QSIPGNEERFDSYPMVLGAQHFHSGKHYWEVDVTG |
| | | KEAWDLGVCRDSVRRKGHFLLSSKSGFWTIWLWNK |
| | | QKYEAGTYPQTPLHLQVPPCQVGIFLDYEAGMVSF |
| | | YNITDHGSLIYSFSECAFTGPLRPFFSPGFNDGGK |
| | | NTAPLTLCPLNIGSQGSTDY |
| 05 | CHIPΔTPR | MRLNFGDDIPSALRIAKKKRWNSIEERRIHQESEL |
| | | HSYLSRLIAAERERELEECQRNHEGDEDDSHVRAQ |
| | | QACIEAKHDKYMADMDELFSQVDEKRKKRDIPDYL |
| | | CGKISFELMREPCITPSGITYDRKDIEEHLQRVGH |
| | | FDPVTRSPLTQEQLIPNLAMKEVIDA FISENGWV |
| | | EDY |

The SARS-CoV-2 receptor binding domain (RBD) peptides/proteins can be tested, either fused to the Fc receptor and co-expressed with an E3 ubiquitin ligase, such as TRIM 21, or directly fused to an E3 ubiquitin ligase, such as CHIPΔTPR.

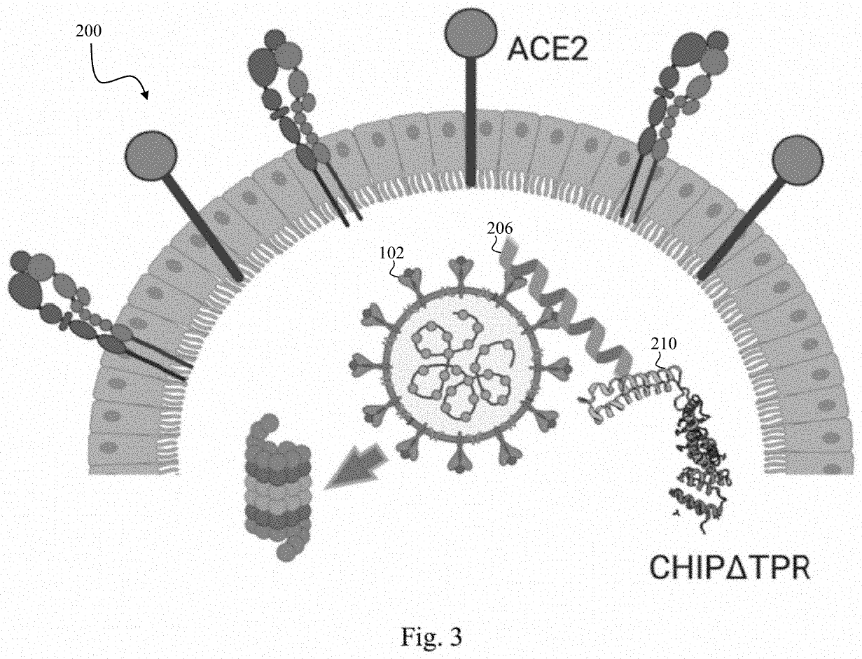

Referring now to FIG. 3, another example mediated degradation 200 of a SARS-CoV-2 virus is shown to demonstrate the function of the fusion peptide in mediating degradation of a target virus. In example 200, RBD 102 is bound by a targeting domain 206 of a fusion protein. The fusion protein comprises the targeting domain 206 fused directly to a E3 ubiquitin ligase, such as CHIPΔTPR shown in example 200.

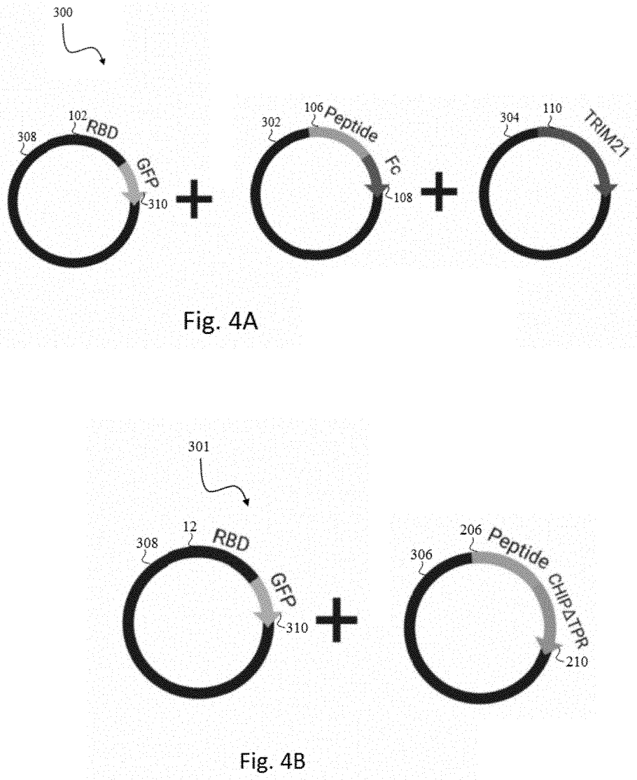

For testing, for example in human HEK293T cells, the SARS-CoV-2 receptor binding domain (RBD) can be fused to GFP, expressed and tested for GFP degradation. FIGS. 4A and 4B are schematic representations of an example experimental outline 300.

Example method 100, from FIG. 1, is shown in FIG. 4A as plasmid 302, comprising targeting peptide 106 and an Fc domain 108, and plasmid 304, comprising an E3 ubiquitin ligase recruitable by the Fc domain 108, such as TRIM 21 110.

Example method 200, from FIG. 3, is shown in FIG. 4B as plasmid 306, comprising targeting peptide 206 and an E3 ubiquitin ligase fused directly to the targeting peptide 206, such as CHIPΔTPR 210.

A plasmid 308 comprising a viral RBD 102 fused to GFP 310 allows measurement of effective attachment and degradation by the fusion peptides of plasmids 302 and 306, as fluorescence produced by GFP 310 will decline as plasmids 308 are bound and degraded.

Example materials and methods for carrying out example verification experiment 300 are as follows. HEK293T cells were maintained in DMEM supplemented with 100 units/ml penicillin, 100 mg/ml streptomycin, and 10% fetal bovine serum (FBS). The RBD-GFP plasmids 308 (333 ng) and peptide plasmids 306 (333 ng) were transfected with the RBD-GFP plasmid 308 (333 ng), and corresponding peptide plasmid 302 (333 ng), and E3-ubiquitin ligase plasmid 304 (333 ng) into cells as duplicates (2×105 per well in a 24-well plate) with Lipofectamine 3000 (Invitrogen) in Opti-MEM (Gibco). After 5 days posttransfection, cells were harvested and analyzed on a NXT Attune Flow Cytometer (Thermo Fisher) for GFP (488-nm laser excitation, 530/30 filter for detection) fluorescence. Cells expressing GFP were gated, and percent GFP+ depletion to the RBD-GFP only control were calculated. All samples were performed in duplicates, and percentage values were averaged. SD was used to calculate error bars.

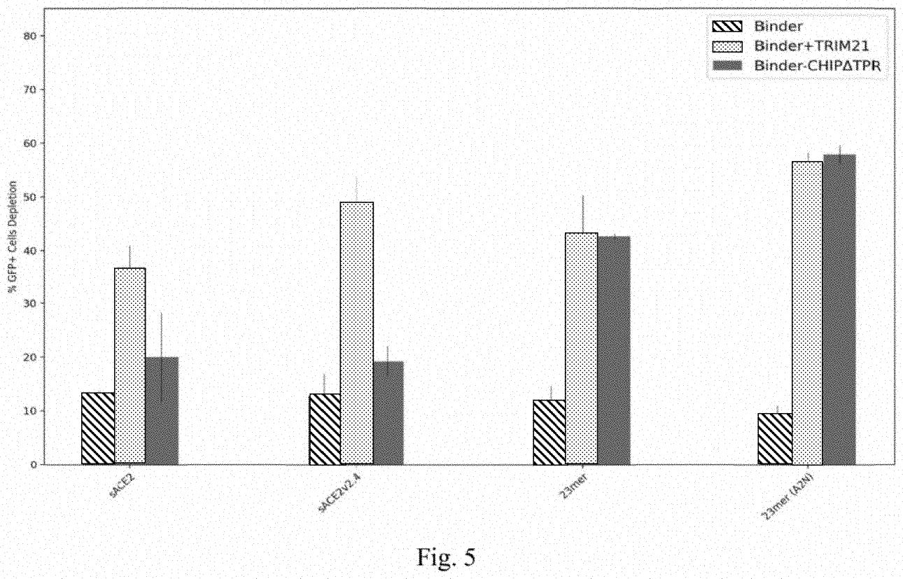

The graph of FIG. 5 summarizes the results of these example tests. The data demonstrates that the 23mer peptide, fused to Fc does demonstrate RBD-GFP degradation when co-expressed with TRIM21. To improve its binding to the RBD, computationally-optimized mutations were introduced into the 23mer, and an A2N (alanine to asparagine at position 2) mutation was identified that demonstrated over 50% depletion of the target protein. Similar levels of degradation are seen when the 23mer (A2N) peptide is fused directly to the E3 Ubiquitin Ligase CHIPΔTPR.

### EXAMPLES

### Example 1: Computationally Optimized Peptides Targeting the SARS-CoV- 2 RBD

Example 1 demonstrates a method for generating and evaluating engineered targeting peptides. This example further assesses whether an RBD-protein targeting peptide, exhibits cross-binding affinity toward previous spike proteins for which a known structure exists, thus allowing determination of the peptides tolerance to viral evolution.

A structure of the SARS-CoV-2 RBD bound to sACE2 was retrieved from the Protein Data Bank (PDB 6M0J). The PeptiDerive protocol in the Rosetta protein modeling software was used to generate truncated linear sACE2 peptide segments between 10 and 150 amino acids with significant binding energy compared to that of the full SARS-CoV-2 RBD-sACE2 interaction. To analyze the conformational entropy of the peptide segments in the binding pocket, both the FlexPepDock and Protein-Protein protocols were employed to dock the peptides to the original RBD. To ensure tolerance to potential mutations in the RBD, peptides were docked with optimal binding energies against the divergent 2003 SARS-CoV RBD bound with ACE2 (PDB 2AJF). Peptides that demonstrated highest binding energy for SARS-CoV and SARS-CoV-2 RBD were then docked against the α5β1 integrin ectodomain (PDB 3VI4) to identify weak off-target binders. Finally, as bounded ligands may alter the native conformation of proteins, the conformational stability of the candidate peptides as monomers was confirmed to rule out any destabilizing factors in their unbound state. After applying these filters, 26 candidate peptides were selected from a total list of 188 initial peptides as disclosed in U.S. patent application Ser. No. 17/222,676.

### Example 2: Targeted Degradation of RBD with a Co-Expressed E3 Ubiquitin Ligase

TRIM 21 is an E3 ubiquitin ligase that binds with high affinity to the Fc domain of antibodies and recruits the ubiquitin-proteasome system to degrade targeted proteins. Recently, the Trim-Away technique was developed for acute and rapid degradation of endogenous proteins, by co-expressing TRIM21 with an anti-target antibody [Clift, D. et al. A method for the acute and rapid degradation of endogenous proteins. Cell 171, 1692-1706.e18 (2017).]. By fusing the Fc domain to the C-terminus of candidate peptides and co-expressing TRIM21, degradation of the RBD fused to a stable fluorescent marker, such as superfolder GFP9 (RBD-sfGFP), in human HEK293T cells can be mediated using a simple plasmid-based assay. Two compact candidate targeting peptides from Example 1 were used, an 18-mer and 23-mer derived from the ACE2 peptidase domain α1 helix, which is composed entirely of proteinogenic amino acids, as well as the candidate peptide computationally predicted to have highest binding affinity to the RBD for testing alongside sACE2. A recently-engineered 23-mer peptide from Zhang, et al., purporting to have strong RBD-binding capabilities [Zhang, G., Pomplun, S., Loftis, A. R., Loas, A. & Pentelute, B. L. The first-inclass peptide binder to the SARS-CoV-2 spike protein. bioRxiv https://doi.org/10.1101/2020.03.19.999318 (2020).], was also tested. Five days post transfection, the degradation of the RBD-sfGFP complex was analyzed by flow cytometry. After confirming negligible baseline depletion of GFP+ signal with and without exogenous TRIM21 expression, as well as no off-target degradation of sfGFP unbound to the RBD, over 30% reduction of GFP+ cells treated with full-length sACE2 fused to Fc and co-expressed with TRIM21 was observed, as compared to the RBD-sfGFP-only control. Of the tested peptides, only the 23-mer demonstrated comparable levels of degradation, with ˜20% reduction in GFP+ cells.

### Example 3: Engineering of an Optimal Peptide-Based Degradation Architecture

Recently, deep mutational scans have been conducted on sACE2 to identify variants with higher binding affinity to the RBD of SARS-CoV-229. Similarly, a complete single point mutational scan was conducted for all 23 positions in the peptide using the ddG-backrub script in Rosetta to identify mutants with improved binding affinity [Barlow, K. A. et al. Flex ddG: Rosetta ensemble-based estimation of changes in protein-protein binding affinity upon mutation. J. Phys. Chem. B 122, 5389-5399 (2018).]. For each mutation, 30,000 backrub trials were performed to sample conformational diversity. The top eight mutations predicted by this protocol were used for an experimental assay, along with the top eight mutations predicted using an Rosetta energy function optimized for predicting the effect of mutations on protein-protein binding, as well as the top eight mutational sites within the 23-mer sequence from deep mutational scans of sACE2. Results in the subsequent TRIM21 assay identified A2N, derived from the original Rosetta energy function, as the optimal mutation in the 23-mer peptide, which achieved over 50% depletion of GFP+ cells, improving on both the sACE2 and 23-mer architecture as well as that of a previously optimized full-length mutant, sACE2v2.4.

### Example 4: Inhibition of Infection-Competent Viral Production

The efficacy of the 23-mer (A2N)-CHIPΔTPR fusion against viruses pseudotyped with the SARS-CoV-2 S protein was assessed. A plasmid encoding the construct was introduced during lentiviral production with a ZsGreen expression plasmid, lentivirus packaging plasmid, and an envelope protein plasmid encoding the full-length S protein, rather than just the RBD. After viral supernatant recovery, HEK293T cells expressing doxycycline-induced hACE2 were infected, and quantified infection as the percentage of ZsGreen+ cells by flow cytometry. Results showed that the 23-mer (A2N)-CHIPΔTPR fusion reduces the infection rate of the pseudovirus by ˜60%, in agreement with our RBD-sfGFP degradation data.

Other possible mechanisms of action for the peptide variant were tested, namely competitive interception of S protein-pseudotyped virus prior to cellular entry. The 23-mer (A2N) peptide was synthesized, and the pseudoviral assay was repeared in its presence or absence at a standard dosage (1 μg/ml) [Monteil, V. et al. Inhibition of SARS-CoV-2 infections in engineered human tissues using clinical-grade soluble human ACE2. Cell 181, 905-913.e7 (2020).]. Minimal difference in pseudoviral infection competency was observed with the addition of the exogenous peptide in all tested experimental conditions, thus suggesting that intracellular delivery of the 23-mer (A2N)-CHIPΔTPR fusion may be the optimal modality for the peptide to inhibit viral infection.

At least the following aspects, implementations, modifications, and applications of the described technology are contemplated by the inventors and are considered to be aspects of the presently claimed invention: (1) A minimal, specific, nucleotide encodable, protein to proteasome linker; (2) A minimal, specific, nucleotide encodable, protein to proteasome linker, comprising a peptide-Fc fusion in which the peptide binds to the specific protein of interest and the Fc domain binds to TRIM21; (3) A minimal, specific, nucleotide encodable, protein to proteasome linker, comprising a peptide-E3 ubiquitin ligase fusion in which the peptide binds to the specific protein of interest; (4) A polynucleotide coding for the peptide-Fc fusion; (5) A polynucleotide coding for the peptide-E3 ubiquitin ligase fusion; (6) Peptide-based therapeutic comprising a polynucleotide coding for the peptide-FC fusion and a polynucleotide coding for the peptide-E3 ubiquitin ligase fusion coupled with a delivery vector which may be either a virus or a micelle; (7) Peptide-based therapeutic comprising a minimal, specific, nucleotide encodable, protein to proteasome linker, comprising a peptide-Fc fusion in which the peptide binds to the specific protein of interest and the Fc domain binds to TRIM21, and a minimal, specific, nucleotide encodable, protein to proteasome linker, comprising a peptide-E3 ubiquitin ligase fusion in which the peptide binds to the specific protein of interest, wherein the peptide fusion is further fused to a cell penetrating motif or a cell surface receptor binding motif.

While certain embodiments of the present disclosure are discussed herein, many other implementations will occur to one of ordinary skill in the art and are all within the scope of the invention. Each of the various embodiments described above may be combined with other described embodiments in order to provide multiple features.

Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

1. An engineered peptide for mediated degradation of a target infectious microbe, comprising:
a fusion protein comprising a targeting domain and a ubiquitin ligase recruiting domain;
wherein the targeting domain is engineered to bind the target infectious microbe for mediation degradation by the ubiquitin-proteosome pathway.

2. The engineered peptide of claim 1, wherein the target infectious microbe is a virus.

3. The engineered peptide of claim 2, wherein the targeting domain binds to a spike protein receptor

4. The engineered peptide of claim 3, wherein the spike protein receptor is part of a viral envelope.

5. The engineered peptide of claim 3, wherein the spike protein receptor is part of a coronavirus.

6. The engineered peptide of claim 5, wherein the coronavirus is SARS-CoV-2.

7. The engineered peptide of claim 3, wherein the targeting domain comprises an sACE2-derived peptide consisting of SEQ. ID. No. 1, an A2N derived peptide consisting of SEQ. ID. No. 2, or an A2N_H11A derived peptide consisting of SEQ. ID. No. 3.

8. The engineered peptide of claim 7, wherein the targeting domain comprises an amino acid sequence being at least 90% identical to a sequence selected from the group consisting of SEQ. ID. No. 1, SEQ. ID. No. 2, and SEQ. ID. No. 3.

9. The engineered peptide of claim 7, wherein the targeting domain comprises an amino acid sequence being at least 80% identical to a sequence selected from the group consisting of SEQ. ID. No. 1, SEQ. ID. No. 2, and SEQ. ID. No. 3.

10. The engineered peptide of claim 1, wherein the ubiquitin ligase recruiting domain is an Fc domain.

11. The engineered peptide of claim 10, wherein the viral receptor binding domain has a C-terminus, and the Fc domain is fused to the C-terminus.

12. The engineered peptide of claim 10, wherein the ubiquitin ligase recruiting domain recruits an E3 ubiquitin ligase.

13. The engineered peptide of claim 12, wherein the E3 ubiquitin ligase is TRIM21, corresponding to SEQ. ID. 4.

14. The engineered peptide of claim 1, wherein the ubiquitin ligase recruiting domain is an E3 ubiquitin ligase.

15. The engineered peptide of claim 15, wherein the E3 ubiquitin ligase is CHIPΔTPR, corresponding to SEQ. ID. 5.

16. A method for the treatment or alleviation of an infection by an infectious microbe in a subject comprising:
administering to the subject an engineered peptide or pharmaceutically acceptable salt thereof, wherein the engineered peptide comprises a fusion of a targeting domain and a ubiquitin ligase recruiting domain; and
wherein the targeting domain is engineered to bind the target infectious microbe for mediation degradation by the ubiquitin-proteosome pathway.

17. The method of claim 16, wherein the targeting domain comprises an engineered sACE2-derived peptide.

18. The method of claim 16, wherein the targeting domain comprises an sACE2-derived peptide consisting of SEQ. ID. No. 1, an A2N derived peptide consisting of SEQ. ID. No. 2, or an A2N_H11A derived peptide consisting of SEQ. ID. No. 3.

19. The method of claim 18, wherein the targeting domain comprises an amino acid sequence being at least 90% identical to a sequence selected from the group consisting of SEQ. ID. No. 1, SEQ. ID. No. 2, and SEQ. ID. No. 3.

20. The method of claim 16, wherein the infection is caused by a coronavirus.


SEQUENCE LISTING (5 sequences)

SEQ ID NO: 1 - protein, 23 aa, Artificial Sequence
  computationally truncated and engineered human sACE2 receptor sequence
QAKTFLDKFNHEAEDLFYQSSLA

SEQ ID NO: 2 - protein, 23 aa, Artificial Sequence
  computationally truncated and engineered human A2N receptor sequence
QNKTFLDKFNHEAEDLFYQSSLA

SEQ ID NO: 3 - protein, 23 aa, Artificial Sequence
  computationally truncated and engineered human A2N_H11A receptor sequence
QNKTFLDKFNAEAEDLFYQSSLA

SEQ ID NO: 4 - protein, 475 aa, Artificial Sequence [sequence omitted]

SEQ ID NO: 5 - protein, 177 aa, Artificial Sequence
  computionally engineered CHIPDTPR E3 Ubiquitin Ligase Sequence
MRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY